(19) United States
(12) Patent Application Publication
(10) Pub. No.: US 2023/0149671 A1
(43) Pub. Date: May 18, 2023

(54) Steerable Guidewire and Method of Use

(71) Applicant: Indian Wells Medical, Inc., Lake Forest, CA (US)

(72) Inventor: Jay Alan Lenker, Lake Forest, CA (US)

(73) Assignee: Indian Wells Medical, Inc., Lake Forest, CA (US)

(21) Appl. No.: 17/986,401

(22) Filed: Nov. 14, 2022

Related U.S. Application Data
(60) Provisional application No. 63/279,104, filed on Nov. 13, 2021.

(51) Int. Cl. A61M 25/09; A61M 25/00; A61M 25/01

(52) U.S. Cl. CPC A61M 25/09041; A61M 25/0097; A61M 25/0138; A61M 25/0147; A61M 2025/0004

(57) ABSTRACT

A steerable endoluminal access device, such as a guidewire or guide catheter. The steerable endoluminal access device includes an inner tube within an outer tube, with the inner tube fixed to the outer tube near the distal end of the device. The steerable endoluminal access device includes a releasably detachable hub for tensioning or compressing the inner tube relative to the outer tube.

This application claims priority to U.S. Provisional Application 63/279,104 filed Nov. 13, 2021.

### FIELD OF THE INVENTION

The inventions described below relate to the field of endovascular access to the cardiovascular system.

### BACKGROUND OF THE INVENTION

During certain interventional procedures that are directed at cardiac access, the patient is catheterized through an access point in a vein or artery. A catheter is routed to the heart or other region of the cardiovascular system through the access point, which may be created by a cutdown or a percutaneous access procedure. The catheter may be routed to a target location within the heart, cerebrovasculature, or other region of the cardiovascular system. The routing is typically performed using a percutaneous access procedure, in some cases called a Seldinger procedure. In other vascular access procedures, open surgical access is required. In either case, an endoluminal access device is advanced into the vasculature by way of the percutaneous or open procedure. The endoluminal access device, when configured as a guidewire, can serve as a tracking system over which a catheter can be routed to a target site within the patient, or through which a catheter can be routed to a target site within the patient. The endoluminal access device, when configured as a guide catheter, can be used for procedures such as transcatheter, endovascular, or vascular access as well as for transcutaneous, laparoscopic, thoracoscopic, and intramuscular access, and the like.

### SUMMARY OF THE INVENTIONS

An endoluminal access device, such as a guidewire or guide catheter, is disclosed wherein the device is capable of articulating, steering, bending, deflecting, or otherwise being controlled off-axis to permit tracking within a vessel or body tissue, or moving to within a certain target location within a hollow organ. This articulation or steering can be performed without interaction with any guide catheters.

In some embodiments, the steerability, deflection, or articulation, of a distal region of the endoluminal access device can be accomplished using the inner tube and outer tube, concentrically arranged and radially constrained together in the distal region of the endoluminal access device. The inner tube outer diameter is a close tolerance fit to the inside diameter of the outer tube but the inner tube is free to translate, except where longitudinally fixed to the outer tube, along a longitudinal axis of the tubes relative to the outer tube. Thus, only translational motion and relative compression or tension of the inner tube relative to the outer tube, along the longitudinal axis, is used to generate the articulation. The inner tube is modified in a region proximate the distal end such that the inner tube is divided, weakened, or split, into two or more parts along a generally longitudinal direction. Only a portion of these divided parts of the inner tube are affixed, at their proximal end, to the more proximal portion of the inner tube. The parts of the inner tube not affixed at their proximal end can be optionally affixed near their distal end to the portions of the inner tube that are also affixed at their proximal end. The outer tube is rendered flexible by cutting slots or gaps generally having a lateral or radial orientation, although there can be some projection at an angle or along the longitudinal axis of the outer tube. These lateral slots do not pass completely through the outer tubing so a spine with ribs is formed in the outer tubing. In other embodiments, the outer tube can be formed as a helix or a coil having a finite spacing between the coils or windings along one or more sides of the distal region. A backbone or series of locking devices can optionally be added to prevent longitudinal compression or expansion on one side of the winding or coil in this configuration. As used herein, the coil construction embodiment having a backbone or fixation column is used interchangeably with embodiments where the distal part of the outer tube which is rendered flexible by way of a plurality of cuts, lateral slots, or the like.

The inner tube can be affixed to the outer tube at a region distal to the lateral slots in the outer tube. The portion of the inner tube that is affixed to the outer tube is that portion of the split inner tube that is connected at its proximal end to the more proximal portions of the inner tube. The inner tube can be configured with an asymmetric distal end. In a preferred embodiment, the inner tube is split along its length at the distal end. The split is oriented so that it radially migrates toward and through the side of the inner tube on one side. This configuration leaves a connected side and a disconnected side to the inner tube. The disconnected side can be affixed to the connected side near the distal end by a bridge.

The inner tube can be a tube or it can be a solid core structure such as a round rod, square rod, C-shaped rod, or similar.

Thus, articulation is generated using a plurality of (two or more) nested, radially constrained, substantially concentric axially translating tubes, wherein a first tube is weakened on one side to increase flexibility and limit final curvature and shape while a second tube is split substantially longitudinally and broken off on one side within the region where the first tube is also weakened. In certain embodiments, both tubes are substantially in place to maintain hoop strength, column strength, kink resistance, and orientation of discreet structures, such as breaks or slots exist within the plurality of tubes.

The proximal parts of the endoluminal access device can comprise an outer tube that is of standard, unbroken cylindrical configuration with one or more intermediate tubes fabricated from cylindrical tubes of different bendability, cylindrical tubes having flexibility enhancing slots cut therein, closed coils, braided windings, or coils being somewhat open between the windings. In other embodiments, the proximal region, the inner tube can be affixed to a solid structure or a wire rather than a tube. In other embodiments, a tether or restraint wire can be used to prevent excessive opening of spring coils. The tether can be affixed distal to the distal end of the coil at one end and proximal to the proximal end of the coil at the other end.

The steerable guidewire or guide catheter can be fabricated in diameters ranging from about 0.010 inches to about 0.038 inches, or larger. In certain embodiments, it may be beneficial to build these devices in outside diameters of 0.050 to 0.080 inches or larger. These larger size track devices can be beneficial in larger vessels, such as, but not limited to, the aorta, iliac arteries, superior and inferior vena cava, femoral veins, and the core chambers of the heart such as the ventricles and atria. They can come in various stiffnesses and tip shapes. The guidewire or catheter can be made available in un-deflected tip configurations such as, but not limited to, a floppy, straight tip, a J-curve tip, and a slight curve tip, for example. The length of a guidewire can range from about 50-cm to about 250-cm or longer. The steerable guidewire can be beneficially, typically twice as long as a catheter which is to be loaded over the guidewire so that the guidewire can remain gripped by the user at both its proximal and distal ends with the catheter fully inserted over the guidewire. A guide catheter can be used in conjunction with the guidewire to achieve some steerability. For example, a guidewire with a curved tip can be withdrawn into a guide catheter to achieve a straight configuration, then be advanced outside the guide catheter into its curve and advanced into a vessel or body lumen, wherein the guide catheter is then advanced along the guidewire until the next steering event is required. The tip curving can, in the preferred embodiments, be generated by articulation in situ, rather than pre-curving.

In other embodiments, the steerable guidewire or catheter can be configured in large sizes to control tip position of large diameter delivery devices such as those used for heart valve replacements, mitral valve repair devices, left atrial appendage occluders, annuloplasty rings, chordae tendineae repair systems, renal artery access systems, and the like. Such systems are not guidewires but delivery systems or introduction sheaths for these structural repair implants, diagnostic, or other therapeutic devices.

The steerable endoluminal access device can be configured with a hub that is permanently attached or it can be configured with a hub that is detachable. In yet another embodiment, the hub can be detachable and re-attachable. In other embodiments the hub is detachable, re-attachable, and able to provide deflecting control over the distal end, following re-attachment.

In detachable embodiments, the hub can be releasably affixed to the inner and outer tubes using pins, collet devices, elastomeric compression fittings (Tuohy-Borst) fittings, clamps, spring-loaded clamps or pins, or other fasteners. These pins, clamps, and the like can engage the inner tube or rod and outer tube by way of diameter increases, protrusions, bosses, roughened areas, circumferential grooves, depressions, bumps, or collars, affixed or integral to the exterior of the tubes or rods. In other embodiments, these pins, clamps, compression fittings, and the like can engage the inner tube or rod and outer tube by way of holes or fenestrations in the tubes. In these embodiments, it is beneficial make the outer tube with an outside diameter smaller that the outside diameter of other tubes, catheters, guide catheters, etc. can be routed over the steerable guidewire after the hub and connecting pieces are removed.

In other embodiments, a control fitting can be disposed between the inner tube compression fitting and the outer tube compression fitting to provide controlled separation. The control fitting can comprise configurations such as, but not limited to, a lever, a cam, threaded knob, an electromechanical actuator, a pistol grip with trigger, and the like. In some embodiments, the control fitting can be integral to either the first compression fitting, the second compression fitting, or both compression fittings. In the embodiment where the control fitting is integral or affixed to both compression fittings, the result is a simple control hub that is removable from the guidewire thus allowing catheters and other devices to be advanced over the guidewire.

In other embodiments, the inner tube or rod and the outer tube can have their relative positions maintained after removal of the hub. This maintenance of position can occur by way of one or more pins that engage holes or fenestrations in the inner and outer tube. These pins can be spring loaded in some embodiments. The pins can include round, square, u-shaped, or other cross-sectional configurations. The pins can be inserted into the central lumen of the inner tube. They can be routed to a window location in the inner tube and secured at that point from longitudinal or axial movement. When the outer tube window or hole is moved to alignment with the hole or window in the inner tube, the pin can be advanced outward through the hole in the outer tube, thus securing the outer tube and inner tube at a specific longitudinal, and optionally radial, relative position. This methodology can allow for securement at discreet locations consistent with the locations of the holes in the outer tube. A plurality of holes in the outer tube can allow for maintenance of distal curvature at more than one configuration. Selective rotational alignment of the inner tube pin relative to the outer tube can be used to position the inner tube pin at intermediate positions with respect to the outer tube.

In yet other embodiments, the proximal end of the inner tube can comprise an external thread on its exterior. The inner tube is longer than the outer tube such that its proximal end extends out beyond the proximal end of the outer tube. A separate collar with an interior thread can be engaged with the threaded inner tube. The control knob on the hub can be releasably affixed to this threaded collar. Rotation of the threaded collar can exert compression on the proximal end of the outer tube. This compression on the outer tube relative to the inner tube can activate the bending or steering capability of the distal end of the inner tube/control rod and outer tube subassembly. Once the hub with control knob is removed from the inner and outer tube subassembly, the collar can remain in place to precisely maintain the curve at the distal end. This collar is preferentially sized such that its outside diameter is approximately the same or less than that of the outer tube outside diameter. This sizing can permit catheters to be routed over the structure in the manner of a guidewire.

In other embodiments, the inner tube can be cut to create a plurality of leaf springs in its surface. Since the inner tube is preferentially a metal with a high modulus of elasticity, e.g. spring temper or spring drawn or rolled, these leaf springs can be configured to bend radially outwardly. These leaf springs can be used to prevent proximal movement of the outer tube relative to the inner tube. Such leaf springs could also be configured to prevent distal movement of the outer tube relative to the inner tube. The outermost radial extent of the leaf springs is preferably not larger than that of the outer tube or the inside diameter of any catheter or dilator that will be threaded over the steerable guidewire. The leaf springs can be compressed radially inward by features on the hub or by separate apparatus. The compressed leaf springs can permit the inner tube and outer tube to slide longitudinally relative to each other. The leaf springs can provide for digital or discreet points of positional locking between the two tubes. The leaf springs can be positioned at various circumferential locations on the inner tube to increase the resolution or difference in locking position between the outer tube and inner tube.

In another embodiment, a twist lock can be created at the hub end of the steerable guidewire. Rotation of the twist lock can prevent relative movement of the inner tube relative to the outer tube. This twist lock can take the form of two rectangular or oval tube segments nested within each other. The inner tube is configured with a dimensional increase at its proximal end. By exposing the dimensionally increased part of the inner tube relative to the outer tube and twisting it to approximately 90 degrees from its nested orientation, the inner tube is prevented from moving within the outer tube. This apparatus can provide for one or more discreet locking location. The twist lock can be free to move or it can be spring loaded to control its circumferential position.

In another embodiment, the steerable guidewire can be used in the manner of a track over which a catheter can be routed without removal of the hub. Such tracking can take the form of a feature on the side of the catheter that permits longitudinally slidable attachment to the guidewire exterior at points distal to the hub. By way of example, a c-clip or openable and closable ring, such as a carabiner, can be affixed to the side of the catheter. The catheter and, optionally its dilator or obturator, can be routed thus, beside the guidewire and not over it, to a target location in the body to which the guidewire has already been placed. The catheter can beneficially include one or more of these sliding clips and the tip of the catheter can be made asymmetrical such that it tapers toward the guidewire and not to a central point on the cross-section of the catheter and its dilator.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1A illustrates a side, partial cutaway view of a steerable guidewire having a hub, a proximal region, an intermediate region, and a steerable distal region, according to an embodiment of the invention;

FIG. 1B illustrates a magnified side, partial breakaway view of a transition between the distal steerable region and the intermediate region, according to an embodiment of the invention;

FIG. 2A illustrates a side, partial breakaway, view of an outer tube of a steerable guidewire comprising a plurality of slots near the distal end to generate a region of increased flexibility, according to an embodiment of the invention;

FIG. 2B illustrates a side, partial breakaway, view of an inner, tube of the steerable guidewire comprising a longitudinal slot dividing the tube into two axially oriented parts which are connected at the distal end of the inner tube, according to an embodiment of the invention;

FIG. 3 illustrates the proximal end of the steerable guidewire comprising a first compression fitting releasably affixed to the outer tube and a second compression fitting releasably affixed to the inner tube, according to an embodiment of the invention; and

FIG. 4 illustrates the first and second compression fittings assembled to the guidewire of FIG. 3, wherein a control mechanism has been assembled between the two compression fittings, according to an embodiment of the invention.

### DETAILED DESCRIPTION OF THE INVENTIONS

In accordance with current terminology pertaining to medical devices, the proximal direction, or end, is defined herein as that direction, or end, on the device that is furthest from the patient and closest to the user, while the distal direction, or end, is that direction, or end, closest to the patient and furthest from the user. These directions and locations are applied along the longitudinal axis of the device, which is generally an axially elongate structure optionally having one or more lumens or channels extending through the proximal end to the distal end and running at least a portion of the length of the device. A catheter size given in the units of “French” or “Fr” is defined as three times (approximately pi) the diameter in millimeters. Thus, a device that is 2 mm in diameter can be said to have a diameter of 6 French.

In an embodiment, the invention is an endoluminally, transvascularly, or endovascularly placed steerable guidewire, with internal deflectability or the ability to articulate, at its distal end, in a direction away from its longitudinal axis. The steerable guidewire is generally fabricated from stainless steel, nitinol, or the like and comprises an outer tube, an inner tube, and a distal articulating region. The deflecting or articulating mechanism is integral to the steerable guidewire. The steerable guidewire is useful for animals, including mammals and human patients and is routed through body lumens or other body structures to reach its target destination.

In an embodiment, the steerable guidewire comprises an inner tube and an outer tube. The steerable guidewire can also comprise a stylet or obturator, which can be removable or non-removable. The steerable guidewire further comprises a hub at its proximal end which permits grasping of the steerable guidewire as well as features, or control mechanisms, for controlling the articulation at the distal end. Such features can comprise control knobs, handles, levers, or the like. The proximal end further can optionally be terminated with a female Luer or Luer lock port or hemostasis valve, which is suitable for attachment of pressure monitoring lines, dye injection lines, vacuum lines, a combination thereof, or the like. The steerable guidewire can comprise a center channel operably affixed to the Luer or Luer lock port, said channel being useful for dye injection, material or fluid administration or removal, pressure monitoring, or the like. In some embodiments, it is beneficial that a catheter be advanceable over the guidewire beginning at the proximal end of the guidewire. In these embodiments, the hub, which comprises controlling mechanisms for the distal deflection, is beneficially detachable from the guidewire and can be releasably or non-releasably affixed to the guidewire following advancement of a catheter past the proximal end of the guidewire.

The steerable guidewire or catheter can be fabricated so that it is substantially straight from its proximal end to its distal end. Manipulation of a control mechanism at the proximal end of the steerable guidewire causes a distal region of the steerable guidewire to bend or curve away from its longitudinal axis. The bending, steering, deflecting, or articulating region is located near the distal end of the steerable guidewire and can be a flexible region or structure placed under tension or compression through control rods or tubular structures routed from the control handle at the proximal end of the steerable guidewire to a point distal to the flexible region.

Other embodiments of the inventions comprise methods of use. One method of use involves inserting the central core wire or stylet so that it protrudes out the distal end of the steerable guidewire. A percutaneous or cutdown procedure is performed to gain access to structures such as, but not limited to, the vasculature, either a vein, an artery, a body lumen or duct, a hollow organ, musculature, fascia, cutaneous tissue, the abdominal cavity, the thoracic cavity, and the like. An introducer, which is usually a hollow, large diameter, hypodermic needle, and the steerable guidewire are placed within the vasculature and the steerable guidewire is routed proximate to the target treatment site. The introducer can be removed at this time or substantially at the time the guidewire is introduced into the body lumen. A guiding catheter, preferably with a removable central obturator or dilator having a core lumen sized to slidably fit over the steerable guidewire, with a tapered distal tip pre-inserted, is routed over the steerable guidewire to the target site. The steerable guidewire can be adjusted so that it assumes a substantially straight configuration. In other procedural embodiments, the steerable guidewire can be advanced through the central lumen of an already placed catheter, sheath, introducer, or guide catheter. The steerable guidewire or catheter comprises a generally atraumatic, non-sharp, distal tip. The distal tip can be rounded, oval, or the like, but can also be sharpened or include electrodes for RF ablation, cryoablation, or HIFU.

The distal end of the steerable guidewire, and optionally the body of the guidewire or catheter as well, is sufficiently radiopaque that it is observable clearly under fluoroscopy or X-ray imaging. The steerable guidewire, especially near its distal end, can be configured with asymmetric radiopaque markers that provide some indication regarding the side of the steerable guidewire that deflection can occur. The location of the steerable guidewire and the amount of deflection and curvature of the distal end are observed and controlled using the aforementioned fluoroscopy or X-ray imaging, or other imaging method such as MRI, PET scan, ultrasound imaging, and the like. The primary structure of the steerable guidewire or catheter shaft is stainless steel or nitinol, or the like. These materials should preferably comprise spring or superelastic temper. A Leak resistant coating can be applied to the exterior or the interior lumen of the inner tube or outer tube. One or more radiopaque markers can be affixed to the distal end of the steerable guidewire to further enhance visibility under fluoroscopy. Such radiopaque markers can comprise materials such as, but not limited to, thick ferrous metals, tantalum, gold, platinum, platinum iridium, and the like.

Deflection of the distal tip to varying degrees of curvature, under control from the proximal end of the guidewire can be performed. The curve can be oriented along the direction of a branching vessel or vessel curve so that the steerable guidewire can then be advanced into the vessel by way of its high column strength and torqueability. Alignment with any curvature of the catheter can be completed at this time. When correctly positioned under fluoroscopy, ultrasound, or other imaging system, dye can be injected into the central lumen of the steerable guidewire at its proximal end and be expelled out of the distal end of the steerable guidewire to provide for road-mapping, etc. This steering function can be very beneficial in device placement and is also especially useful in highly tortuous vessels or body lumens which may further include branching structures such as bifurcations, trifurcations, and the like.

In some embodiments, the inner tube, the outer tube, or both can have slots imparted into their walls to impart controlled degrees of flexibility. The slots can be configured as “snake cuts” to form a series of ribs with one or more spines. The spines can be oriented at a given circumferential position on the outer tube, the inner tube, or both. The spines can also have non-constant orientations. In some embodiments, only the outer tube is slotted. The slots can be generated within the distal portion of the outer tube where the curve is generated. This distance can range between about 0.5-cm and 15-cm of the end and preferably between 1-cm and 5-cm of the distal end. The slot widths can range between 0.001 inches and 0.100 inches with a preferable width of 0.003 to 0.010 inches. In exemplary embodiments, the slot widths are about 0.008 inches. In some embodiments, it is desirable to have the outer tube bend in one direction only but not in the opposite direction and not in either lateral direction. In this embodiment, cuts can be made on one side of the outer tubing within, for example, the distal 10-cm of the tube length. Approximately 5 to 30 cuts can be generated with a width of approximately 0.010 to 0.040 inches. The cut depth, across the tube diameter from one side, can range between 0.1 and 0.9 of the tube diameter. In an embodiment, the cut depth can be approximately 0.4 to 0.6 of the tube diameter with a cut width of 0.025 inches. A second cut can be generated on the opposite side of the tube wherein the second cut is approximately 0.005 inches or less. In an embodiment, the outer tube can be bent into an arc first and then have the slots generated such that when the tube is bent back toward the 0.005 inch wide cuts, the tube will have an approximately straight configuration even through each tube segment between the cuts is slightly arced or curved.

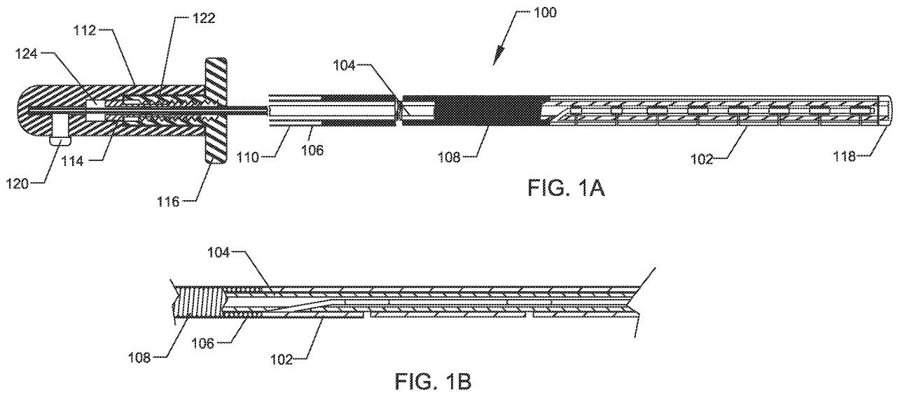

FIG. 1A illustrates a side view of a steerable guidewire 100 comprising an outer tube with distal segment 102 of the outer tube, an intermediate segment 108 of the outer tube, and a proximal segment 110 of the outer tube, and an inner tube 104. The steerable guidewire may also include an outer low-friction coating 106. The steerable guidewire includes a hub 112 further comprising a hub body lumen 124, a jackscrew traveler 114, a control knob 116, an inner tube lock 120, and a proximal outer tube lock 122. The guidewire can include a rounded distal tip 118, or the distal end of the guidewire can terminate in an open lumen, leaving the lumen of the inner tube open so that the device can function as a guide catheter.

Referring to FIG. 1A, the distal end of the inner control tube or rod, hereafter called the inner tube 104, is longitudinally affixed to the distal end of the distal outer tube 102. The inner tube or control rod 104 is slidably disposed within the inner lumen of the distal outer tube 102 except at the distal end where they are affixed to each other. The proximal end of the distal segment of the outer tube 102 is affixed to the distal end of the intermediate segment 108 of the outer tube. The proximal end of the intermediate segment 108 of the outer tube is affixed to the distal end of the proximal segment 110 outer tube. The entire outer tube assembly (segments 102, 108, 110) can be covered with an optional anti-friction coating or layer 106. A guidewire tip 118, if used, maybe affixed to the inner tube 104, the distal outer tube 102, or both. The guidewire tip 118 can comprise a through hole to permit infusion of fluids therethrough or for advancement of a stylet (not shown) beyond the distal end of the guidewire 100. The control knob 116 is rotationally free to move within the hub body 112, to which it is longitudinally affixed and the two components do not move axially relative to each other. The jackscrew traveler 114 can move axially within a lumen 124 within the hub body 112 within the constraints of the end of the internal lumen 124 of the hub body 112. The jackscrew traveler 114 is keyed within the lumen 124 by a non-round cross-section that impinges on complimentary structures within the lumen 124 to prevent relative rotational movement of the two components 112, 124. The jackscrew traveler 114 comprises external threads that are complimentary and fit within internal threads of the control knob 116. Thus, when the control knob 116 is turned, the jackscrew traveler 114 is forced to move axially either forward or backward because the control knob 116 is longitudinally affixed within the hub body 112.

A thread pitch for the jackscrew traveler 114 and the control knob 116 can range from about 16 to about 64 threads per inch (TPI) with a preferred range of about 24 TPI to about 48 TPI and a more preferred range of about 28 to about 36 TPI.

In some embodiments, the hub assembly is removable from the steerable guidewire so that the proximal end of the steerable guidewire 100 retains the same (or smaller) diameter or profile as the intermediate and distal ends of the guidewire. In these embodiments, catheters, guide catheters, introducers, sheaths, or other axially elongate medical devices comprising an internal guidewire lumen can be slipped over the proximal end of the steerable guidewire and advanced into the patient over an already placed steerable guidewire. This embodiment, or approach, provides for catheter exchange, replacement, swapping, or the like. Once the catheter is advanced such that its proximal end is located distal to the proximal end of the steerable guidewire, the hub assembly can be releasably affixed to the proximal end of the steerable guidewire so that the distal end of the guidewire can be deflected under control at the proximal end. The hub assembly illustrated in FIG. 1A provides an outer tube lock 122 and inner tube lock 120 to secure the outer tube 110 and inner tube 104 of the steerable guidewire such that the hub is affixed and in control of the relative axial position of the two tubes. The outer tube lock 122 can be configured as a bayonet mount (as illustrated) or it can comprise a locking button, locking clamp, threaded lock, or the like.

FIG. 1B illustrates a magnified view of the steerable guidewire 100 of FIG. 1A at the transition between the distal end of the intermediate segment 108 of the outer tube and the proximal end of the distal, steerable region. The steerable guidewire 100 transition region comprises the intermediate segment 108 of the outer tube, the distal segment of the outer tube 102, the inner tube 104, and the polymeric outer coating 106.

The polymeric outer coating 106 is optional but beneficial and can comprise materials such as, but not limited to, fluoropolymers such as PTFE, PFA, FEP, polyester, polyamide, PEEK, and the like. The polymeric outer coating 106 can render the coiled embodiment of the intermediate segment 108 of the outer tube, as illustrated, to retain a relatively smooth exterior surface and provide for friction reduction which is useful when passing a long, slender guidewire through a long, catheter lumen. The distal segment 102 of the outer tube can be affixed to the intermediate segment 108 of the outer tube by means of a weld, fastener, adhesive bond, embedment with polymeric, metallic, or ceramic materials, or the like. The intermediate segment 108 of the outer tube, illustrated in this embodiment as a coil structure with substantially no spacing between the coils, is highly flexible and the flexibility can be controlled by the elastic modulus, thickness, and other material properties of the outer coating 106. The intermediate segment 108 of the outer tube, in other embodiments, can comprise structures such as, but not limited to, an unperforated or unfenestrated tube, a tube with partial lateral cuts, a spiral cut tube, a ribcage with a backbone, or the like.

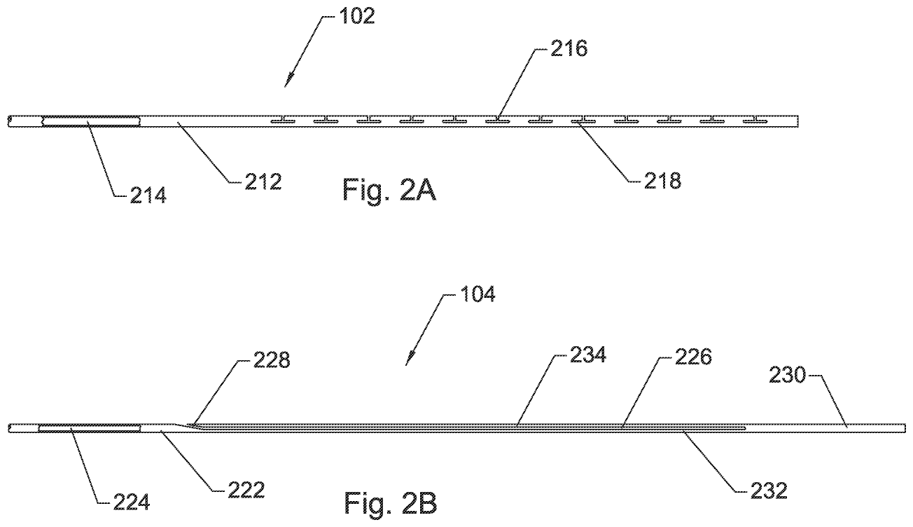

FIG. 2A illustrates a side view, in partial breakaway, of the distal segment 102 of the outer tube, comprising a lumen 214, a proximal, uncut portion 212, a plurality of lateral partial cuts 216, and a plurality of longitudinal “T” or “H” cuts 218, according to an embodiment. The longitudinal portions of the “T” or “H” cuts serve to distribute stresses and allow for more even bending of the tubes off-axis with reduced tendency to yield.

Referring to FIG. 2A, the distal segment 102 of the outer tube serves as the outer tube of the steerable guidewire such as that illustrated in FIG. 1. The plurality of partial lateral cuts 216 serve to render the region of the outer tube 102 in which the lateral cuts 216 are located more flexible than the proximal region 212. The plurality of longitudinal “T” cuts, serve to further render the region of the outer tube 102, in which the “T” cuts 218 reside, more flexible than in tubes where such “T” cuts 218 were not present. The longitudinal “T” cuts 218 are optional but are beneficial in increasing the flexibility of the outer tube 102 in the selected bend region. The partial lateral slots 216 can be spaced apart by about 0.02 to about 1.0 inches with a preferred range of about 0.1 inches to about 0.8 inches and a further preferred range of about 0.15 inches to about 0.5 inches. In an exemplary embodiment, the partial lateral slots 216 are spaced about 0.17 inches apart. The spacing between the partial lateral slots 216 can vary. In some embodiments, for example, the spacing between the partial lateral slots toward the proximal end of the outer tube 102 can be about 0.3 inches while those partial lateral slots 216 nearer the distal end of the outer tube 102 can be spaced about 0.15 inches apart. The spacing can change in a step function, it can change gradually moving from one end of the outer tube 102 to the other, or it can increase and decrease one or more times to generate certain specific flexibility characteristics. Increased spacing increases the minimum radius of curvature achievable by compression of the partial lateral slots 216 while decreased spacing allows for a smaller minimum radius of curvature.

FIG. 2B illustrates an embodiment of a side view, in partial breakaway, of the distal end of an axially elongate inner tube 104, comprising a lumen 224, a proximal, uncut portion 222, a longitudinal slot 226 further comprising an angled lead in 228, a free side 234, a pusher or connected side 232, and a distal tip 230.

Referring to FIG. 2B, the distal tip 230 interconnects the free side 234 and the pusher side 232. The distal tip 230 or end of the inner tube 104 can further comprise a rounded, tapered, or blunted tip or nose cone (not shown). The disconnected free side 234 and the connected pusher side 232 are generally integrally formed but can also be affixed to each other by welding, adhesives, fasteners, or the like. The inner tube may also include partial lateral slots in the longitudinal portion of the inner tube intersected by the longitudinal slot 226, or in the free side 234, to increase flexibility of the inner tube in this region.

As described in relation to the previous figures, the flexible region of the inner tube is disposed within the longitudinal extent of the flexible region of outer tube. In the illustrated embodiment, that slotted portion of the inner tube defines the flexible region of the inner tube, and the snake cut segment of the outer tube defines the flexible region of the outer tube.

The number of lateral cuts 216 or, optionally, the number of lateral cuts 216 with T-cuts 218 can number between about four and about 50 with a preferred number being between about six and about 25 and a more preferred number of about eight to about fifteen. In the illustrated embodiment, there are 12 partial lateral cuts 216, each modified with a “T” or “H”-shaped slot 218. In other embodiments, the partial lateral cuts 216 can be shaped differently. For example, the partial lateral cuts 216 can be at angles other than 90 degrees to the longitudinal axis, curved, V-shaped, Z-shaped, W-shaped or the like. In other embodiments, the ‘T’ slots 218 can have, for example, further cuts approximately lateral to the longitudinal axis, along any portion of the “T” cut 218. This construction provides the outer tube with a flexible region at its distal end. The flexible region is a region at the distal end of the outer tube that is significantly more flexible and susceptible to deflection than the remaining proximal region of the outer tube.

The distal segment 102 of the outer tube can have an outer diameter of about 0.010 to about 0.1 inches with a preferred outside diameter of about 0.015 to about 0.050 inches and a more preferred diameter of about 0.020 inches to about 0.035 inches. In the illustrated embodiment, the outside diameter is about 0.048 inches while the inner diameter is about 0.036 inches. The inside diameter of the distal segment 102 of the outer tube can range from about 0.0.005 inches to about 0.090 inches.

The lead in 228 to the longitudinal slot 226 is beneficially angled to prevent other guidewires, stylets, or other devices, which are inserted through the central lumen 224 from being caught or bumping against an edge. The angled lead in 228 serves a guide to assist with traverse of a stylet, obturator, or guidewire past the lead in 228 and into the distal region of the steerable guidewire. The lead in 228 can be angled from between about −80 degrees (the angle can be retrograde) from the longitudinal axis (fully lateral) to about +2 degrees and preferably from about +5 degrees to about +20 degrees with a most preferred angle of about +8 degrees and about +15 degrees. In the illustrated embodiment, the angle of the lead in slot 228 is about 10 degrees from the longitudinal axis. A second feature of the lead in 228 is that it be positioned or located proximally to the most proximal “T” slot 218 in the outer tube 102 when the two tubes 102, 104 are affixed to each other (see FIG. 9). The lead in 228 is located at least 1-cm proximal to the proximal most “T” slot 218 and preferably at least 2-cm proximal to the proximal most “T” slot 218 so that bending in the distal region does not distort the lead in 228 and cause kinking, misalignment, or pinching of the internal lumen 224.

The inner tube 104 can have an outside diameter that is slightly smaller than the inside diameter of the outer tube 102 so that the inner tube 104 can be constrained to move longitudinally or axially within the outer tube 102 in a smooth fashion with relatively little force exerted. In the illustrated embodiment, the outside diameter of the inner tube 104 is about 0.033 inches giving about a 0.0015 inch radial clearance between the two tubes 102 and 104. The inside diameter of the inner tube 104 can range from about 0.006 to about 0.015 inches less than the outside diameter of the inner tube 104. In the illustrated embodiment, the wall thickness of the intermediate tube is about 0.006 inches so the inside diameter of the intermediate tube is about 0.021 inches. The lumen 224 of the inner tube 104 can be sized to slidably accept a stylet or obturator 140 such as illustrated in FIGS. 1 and 2. A typical stylet wire 140 can range in diameter from about 0.01 to about 0.23 inches with a preferred diameter range of about 0.012 to about 0.020 inches. In another embodiment, the outer tube 102 has an outside diameter of about 0.050 inches and an inside diameter of about 0.038 inches. In this embodiment, the inner tube 104 has an outside diameter of about 0.036 inches and an inside diameter of about 0.023 inches. The radial wall clearance between the inner tube 102 and the outer tube 104 is about 0.001 inches and the diametric clearance is about 0.002 inches. The annulus between the two tubes must be substantially smooth, free from burrs, and free from contamination because the two tubes 102, 104 beneficially need to translate along their longitudinal axis relative to each other over relatively long axial distances of about 50 to about 150-cm.

The inner tube 104 transmits force along its proximal non-slotted region 222 from the proximal end of the inner tube 104 to the lead in 228 where the force continues to be propagated along the connected side 232 to the distal end 230. The outer tube 102 transmits force along its proximal non-slotted region 212. Longitudinal forces applied to the distal, flexible region with the slots 216 cause deformation of the outer tube in an asymmetrical fashion with the side of the outer tube 102 comprising the partial lateral slots 216 forming an outer curve if the slots 216 are expanded and an inside curve if the slots 216 are compressed. Forces to cause bending are preferably exerted such that the partial lateral slots 216 are compressed up to the point where the gap closes, but no further, however forces can also be exerted to expand the slots 216, however limits on curvature are not in place because the lateral slots 216 can open in an unrestrained fashion except for the material properties of the outer tube 102.

The disconnected side 234 of the inner tube 104, separated from the connected side 232 by the longitudinal slot 226 and the lead in 228, serves to maintain an undistorted tube geometry and provide resistance to deformation while helping to maintain the inner lumen 224 in a round configuration and provide a shoehorn or funnel effect to guide an obturator, guidewire, or stylet 140 therethrough as they are advanced distally. The disconnected side 234, being separated from the force transmitting member 222 cannot provide any substantial longitudinal load bearing structure, although at its distal end, where it is integral or affixed to the distal end 230, some tension load carrying capability exists. The inner tube 104 can be considered a split tube and does not carry a load in compression or tension along substantially the entire length of the disconnected side 234. A main advantage of keeping the disconnected side 234 is to maintain the off-center positioning of the force transmitting member 222.

The partial lateral slot 216 in the inner, or intermediate, tube 104 and the T-Slot 218 in the outer tube 102, as well as the longitudinal slot 226 in the inner tube 104, and the lead in slot 228 can be fabricated by methods such as, but not limited to, electron discharge machining (EDM), wire EDM, photo chemical etching, etching, laser cutting, conventional milling, or the like. In other embodiments, different slot configurations can also be employed, such as curved slots, complex slots, zig-zag slots, or the like. In some embodiments, the partial lateral slot 216 can be configured with a tongue and groove or dovetail design to prevent or minimize lateral movement or torqueing of the outer tube 102 in the flexible region. In some embodiments, the tongue and groove or dovetail (not shown) can be generally centered between two “T” slots, for example. The parts can be ganged and fixtured such that, using wire EDM, for example, a plurality of tubes can be cut to reduce manufacturing costs. As many as 20 to 30 tubes, or more, can be fixtured, secured, and etched by the aforementioned methods.

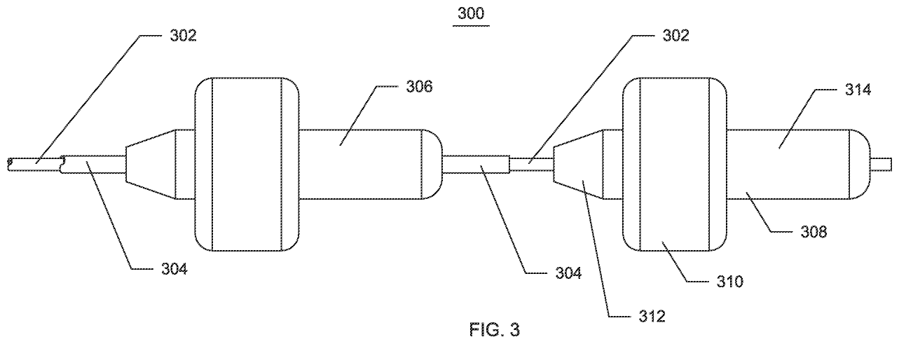

FIG. 3 illustrates the proximal (handle or hub) end of a steerable guidewire system 300 comprising an inner tube 302, an outer tube 304, a first hub fitting 306 comprising a hub body 314, a compression knob 310, and a compression mechanism 312. The steerable guidewire 300 also comprises a second hub fitting 308 comprising a hub body 314, a compression knob 310, and a compression mechanism 312.

Referring to FIG. 3, the first hub fitting 306 is releasably affixed to the outer tube 304 by the compression fitting 312, which operates by means such as, but not limited to, a compression mechanism such as an elastomeric gasket which can be squeezed radially inward to grab the outer tube 304 or the inner tube 302. In other embodiments, the compression mechanism can comprise a collet assembly, a jamb cleat, a toggle clamp, a magnetic coupling, a spring loaded clip that seats in a detent (not shown) on the inner tube 302 or outer tube 304, or the like. The compression fitting 312 can be engaged or disengaged by rotating the compression knob 310, a lever, an electromechanical clamp, or the like, to cause inward radial compression on an engagement mechanism. The second hub fitting 308 is releasably affixed to the inner tube 306 by the same means as that of the first hub fitting 306. The inner tube 306 is smaller in outer diameter than the lumen of the outer tube 304 and is radially constrained to slide axially within the outer tube 304.

In embodiments using compression fittings, a first compression fitting, such as a collet or Tuohy-Borst compression fitting can be affixed to the proximal end of the outer tube and a second compression fitting can be affixed to the proximal end of the inner tube, which beneficially protrudes out the proximal end of the outer tube when the system is unstressed. Thus, the user can move the inner tube axially relative to the outer tube. The axial forces needed to cause tip deflection on these small diameter devices need not be great so simple hand movement can be used to adjust the tip deflection. A Tuohy-Borst compression fitting may comprise a compressible sleeve, distal cap (at item 312) and proximal cap (at 310) both disposed about the compressible sleeve, with the proximal cap and distal cap disposed on either side of the compressible sleeve and configured with internal and external threads so that the threads one cap can engage the threads of the other and the caps may be rotated relative to each other to compress the compressible sleeve and force it to deform radially inwardly to securely engage the tube on which it is disposed. The compression fittings may be configured, as in FIG. 3, to be manipulable by hand to tension or compress the inner tube relative to the outer tube to cause deflection of the distal end of the device. The compression fittings are also preferably configured for releasable attachment and detachment from the tubes, allowing easy attachment and detachment without the use of tools.

The method of using the steerable guidewire can comprise the user assembling the hub fittings to the tubes of the steerable guidewire and then clamping the hub fittings in place using the locking or compression mechanisms. The user can next grasp the hub body 314 of the first hub fitting 306 and the hub body 314 of the second hub fitting 308. By moving (manually or by means of an actuator) the first hub fitting 306 axially relative to the second hub fitting 308, the user can articulate the distal end of the steerable guidewire. Moving the first hub fitting 306 closer to the second hub fitting 308 causes the distal end of the steerable guidewire to deflect in one direction out of the long axis of the steerable guidewire. Moving the first hub fitting 306 away from the second hub fitting 308 causes the distal end of the steerable guidewire to move in a direction opposite that of when the first hub fitting is moved closer to the first hub fitting. Circumferential orientation of the steerable guidewire 300 distal end can be achieved by rotating or torqueing the guidewire about its longitudinal axis. The steerable guidewire 300 of this construction retains the ability to transmit rotation applied to the hub all the way to the distal end.

After the steerable guidewire is routed to its end location, both the first hub fitting 306 and the second hub fitting 308 can be removed from their respective tubes 304 and 302 by loosening the compression mechanisms 312 by way of rotating the knobs 310 and pulling the first 306 and second hub 308 fittings axially off of the proximal end of the steerable guidewire 300. At this point, lumens of other catheters (not shown) can be routed over the proximal end of the steerable guidewire 300 and on to the target location.

The proximal ends of the inner tube 302 and the outer tube 302 can be roughened, finished with circumferential grooves, or the like, to enhance the grip of the compression mechanisms 312 of the hub fittings 306 and 308, while still allowing for loosening and release of the hub fittings 306 and 308.

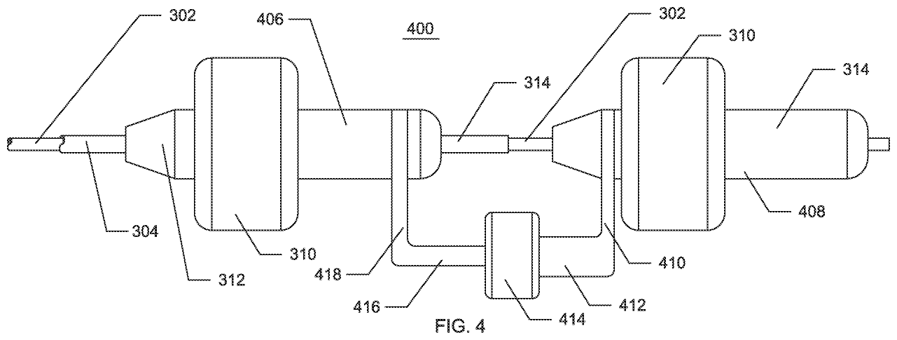

FIG. 4 illustrates the proximal end of a steerable guidewire system 400 comprising the inner tube 302, the outer tube 304, a first hub fitting 406 comprising the hub body 314, the compression knob 310, and the compression mechanism 312. The steerable guidewire system 400 also comprises a second hub fitting 408 comprising the hub body 314, the compression knob 310, and the compression mechanism 312.

The steerable guidewire system 400 further comprises a separation adjusting mechanism which controls the distance between the first hub fitting 406 and the second hub fitting 408. The separation adjusting mechanism comprises the distal fixation device 418, the proximal fixation device 410, a distal linkage 416, a proximal linkage 412, and a control mechanism 414.

Referring to FIG. 4, the distal fixation device 418, the proximal fixation device 410, or both, can be affixed to the first and second hub fittings 406, 408 or they can be integral thereto. The proximal and distal fixation devices can be affixed or integral to the proximal linkage 412 and the distal linkage 416. The proximal and distal linkages 412, 416 are maintained axially in position relative to each other by the control mechanism 414. In the embodiment shown the control mechanism 414 can be a jackscrew. In other embodiments, the control mechanism 414 can be a stepper motor, a cam system, a lever system, a pistol grip with a trigger, a forcep grip expander, or the like. The control mechanism 414 can maintain spacing and allow for precise distal end articulation. Under the precise movement generated by a stepper motor or other electromechanical type actuator, the spacing between the proximal and distal linkages 412, 416 can be controlled by a computer or a computer assisted manual actuation.

However, a tubular or cylindrical central control device can maintain its structure in compression, maintain circumferential location within the outer cylindrical, axially elongate tube, maintain precise control, maintain sufficient tensile strength to exert forces, and maintain a central lumen larger than any other type of steerable device. The resistance to buckling occurs even when the inner tube is slotted longitudinally because the inner tube is constrained within the outer tube using very tight tolerances that will not let the inner tube bend out of its straight orientation, even under compression.

Intermediate sections of the tubes 302 and 304 can comprise coils, snake cut structures with or without backbones, or the like. The use of coils can be facilitated by a tensioner wire disposed through a part or all of the coil area to prevent excessive opening of the coils and thus allow for tensile strength as well as compressive strength when the coils close. Such intermediate tube sections can facilitate routing through tortuous vasculature or body lumens.

A steerable guidewire, as disclosed herein, can be used to route very large, stiff catheters through vasculature of body lumens such that orientation changes can be made without the large diameter, stiff catheter causing the guidewire to straighten out or lose its track because the guidewire can impart its own forces to maintain its curvature. The curvature can be controlled to be small, such as tip deflection, to large and arc shaped, for example to traverse the aortic arch, or turn a 180 degree angle to approach the mitral valve superiorly using an inferior approach through a femoral vein.

The present invention may be embodied in other specific forms without departing from its spirit or essential characteristics. The described embodiments are to be considered in all respects only as illustrative and not restrictive. For example, guidewires can have very similar diameters as the diameters of microcatheters. Thus, the structures and methods disclosed herein can be identically applied to a microcatheter. The forces are very small and compression fittings could easily grasp the movable parts of a microcatheter and pull an inner tube relative to an outer tube. This, of course blurs the line between a microcatheter and a guidewire, the difference being primarily that a microcatheter attempts to maximize the central lumen and this is not so important in a guidewire, if at all. The scope of the invention is therefore indicated by the appended claims rather than the foregoing description. All changes that come within the meaning and range of equivalency of the claims are to be embraced within their scope.

We claim:

1. A steerable endoluminal access device comprising:
an outer tube characterized by a proximal end, a distal end, and a flexible region at said distal end of the outer tube, said flexible region also characterized by a proximal and a distal end; and
an inner tube characterized by a proximal end and a distal end, and a flexible region near said distal end of the inner tube;
said inner tube being disposed within the outer tube, extending from the proximal end of the outer tube to the distal end of the outer tube, and terminating distally proximate the distal end of the outer tube, said inner tube longitudinally fixed to the outer tube at a point in the outer tube proximate the distal end of the flexible region of the outer tube; and
a hub assembly comprising a first compression fitting disposed about the proximal end of the outer tube and a second compression fitting disposed about the proximal end of the inner tube, said first compression fitting operable to be longitudinally fixed to the outer tube and said second compression fitting operable to be longitudinally fixed to the inner tube.

2. The steerable guidewire of claim 1 wherein:
the flexible region of the outer tube comprises a segment of the outer tube which is snake-cut with a plurality of radially oriented slots in the wall of the outer tube, said radially oriented slots being radially aligned along one side of the outer tube; and
the flexible region of the inner tube comprises a segment of the inner tube with a longitudinally oriented slot, wherein said longitudinally oriented slot divides the flexible region of the inner tube into a first partial cylinder segment and a second partial cylinder segment.

3. The steerable guidewire of claim 1 wherein:
a separation adjusting mechanism which controls the distance between the first compression fitting and the second hub fitting.

4. A method of accessing a lumen of a patient with an endoluminal access device, said method comprising the steps of:
providing an endoluminal access device comprising:
an outer tube characterized by a proximal end, a distal end, and a flexible region at said distal end of the outer tube, said flexible region also characterized by a proximal and a distal end; and
an inner tube characterized by a proximal end and a distal end, and a flexible region near said distal end of the inner tube;
said inner tube being disposed within the outer tube, extending from the proximal end of the outer tube to the distal end of the outer tube, and terminating distally proximate the distal end of the outer tube, said inner tube longitudinally fixed to the outer tube at a point in the outer tube proximate the distal end of the flexible region of the outer tube;
inserting the endoluminal access device into a lumen of the patient and navigating the distal end of the endoluminal access device toward a target site within the patient;
securing a first compression fitting about the proximal end of the outer tube such that the first compression fitting is longitudinally fixed to the outer tube;
securing a second compression fitting about the proximal end of the inner tube such that the second compression fitting is longitudinally fixed to the inner tube; and
manipulating the first compression fitting and second compression fitting to tension or compress the inner tube relative to the outer tube to cause deflection of the distal end of the endoluminal access device.
